(19) United States
(12) Patent Application Publication
(10) Pub. No.: US 2003/0113778 A1
(43) Pub. Date: Jun. 19, 2003

(54) Enhanced in vitro nucleic acid synthesis using nucleoside monophosphates

(76) Inventors: Jennifer Theresa Schulte, Stanford, CA (US); James Robert Swartz, Menlo Park, CA (US)

Correspondence Address: BOZICEVIC, FIELD & FRANCIS LLP, 200 MIDDLEFIELD RD, SUITE 200, MENLO PARK, CA, 94025, US

(21) Appl. No.: 10/284,849

(22) Filed: Oct. 30, 2002

Related U.S. Application Data
(60) Provisional application No. 60/335,077, filed on Oct. 30, 2001.

(51) Int. Cl. C12Q 1/68; C07H 21/04; C07H 21/02; C12P 19/34; C12N 9/12

(52) U.S. Cl. 435/6; 435/91.2; 435/194; 536/23.1

(57) ABSTRACT

Compositions and methods are provided for the enhanced in vitro synthesis of nucleic acids. Nucleoside monophosphates are converted to nucleoside triphosphates by in situ reactions to continuously supply the required nucleoside triphosphates. This approach provides for more homeostatic reaction conditions and lower costs.

### BACKGROUND OF THE INVENTION

The directed synthesis of biological macromolecules is one of the great achievements of biochemistry. The development of recombinant DNA techniques has allowed the characterization and synthesis of highly purified coding sequences, which in turn can be used to produce highly purified proteins, even though in native cells the protein may be available only in trace amounts. The biological synthesis may be performed within the environment of a cell, or using cellular extracts and coding sequences, or using systems of purified enzymes to synthesize nucleic acids, proteins, and other macromolecules in vitro.

In vitro techniques allow greater control over reagents and catalytic systems in order to improve productivity, reduce costs and improve product quality. For example, the in vitro synthesis of nucleic acids is useful for producing a broad variety of agents for direct use as therapeutics or for use as templates for the synthesis of additional valuable products.

In addition, In vitro transcription finds particular use when coupled with translation. Because it is essentially free from cellular regulation of gene expression, in vitro protein synthesis has advantages in the production of cytotoxic, unstable, or insoluble proteins. The over-production of protein beyond a predetermined concentration can be difficult to obtain in vivo, because the expression levels are regulated by the concentration of product. The concentration of protein accumulated in the cell generally affects the viability of the cell, so that over-production of the desired protein is difficult to obtain. In an isolation and purification process, many kinds of protein are insoluble or unstable, and are either degraded by intracellular proteases or aggregate in inclusion bodies, so that the loss rate is high.

Recent publications have discussed many different strategies for cost reduction of in vitro transcription reactions, including reusing DNA templates and employing fed batch protocols. For example, see Kern and Davis (1997) “Application of Solution Equilibrium Analysis to in-Vitro RNA Transcription” Biotechnology Progress 13:747-756; Kern and Davis (1999) “Application of a Fed-Batch System to Produce RNA by In-Vitro Transcription” Biotechnology Progress 15:174-184. Their work suggests that pH, free magnesium ion concentration, concentration of pyrophosphate, concentration of individual NTPs, and ionic strength are most important for full length RNA formation. They further suggest that it is the low free magnesium concentration that is limiting transcription, instead of presence of pyrophosphate. Yin and Carter (1996) Nucleic Acids Res. 24(7):1279-86 studied the yield of tRNA obtained from in vitro T7 RNA polymerase transcription using incomplete factorial and response surface methods. The concentrations of T7 RNA polymerase, DNA template, NTP and MgCl₂ proved to be significantly correlated with the yield of tRNA(Trp).

Improvements are required to optimize in vitro transcription systems. The continuous removal of the inhibitory by-product(s) as well as the continuous supply of substrates for nucleic acid synthesis may enable the continuous or semicontinuous reaction system to support synthesis over long reaction periods. However, this approach may also result in inefficient use of substrates and therefore in high costs. Elucidation of inhibitory products, and prevention of their synthesis is of great interest for development of in vitro synthetic systems. Also important is the reduction of reagents costs. With present technology, the major reagent costs include enzymes, DNA template, and NTPs. Methods of decreasing these costs while enhancing yield are of great interest.

Relevant Literature

In vitro transcription has been described in the literature, typically using a bacteriophage RNA polymerase (for example from T7, T3, or SP6 phages). In addition to the RNA polymerase; DNA, magnesium ions, and nucleotide triphosphates are included in the reaction. Additional reagents buffer the pH and inhibit RNases that degrade RNA. There have been various reports of reagent compositions; particularly variations in the concentrations of NTPs and magnesium ions. For example, Milburn et al., U.S. Pat. No. 5,256,555 discloses the use of higher nucleotide concentrations than many other sources, in order to maintain a lower magnesium concentration.

Cunningham and Ofengand (1990) Biotechniques 9:713-714 suggest that adding inorganic pyrophosphatase results in larger reaction yields by hydrolyzing the pyrophosphate that accumulates. Pyrophosphate is inhibitory because the pyrophosphate complexes with the free magnesium ions leaving less available for the transcription reaction.

Breckenridge and Davis (2000) Biotechnology Bioengineering 69:679-687 suggest that RNA can be produced by transcription from DNA templates immobilized on solid supports such as agarose beads, with yields comparable to traditional solution-phase transcription. The advantage of immobilized DNA is that the templates can be recovered from the reaction and reused in multiple rounds, eliminating unnecessary disposal and significantly reducing the cost of the DNA template.

U.S. Pat. No. 6,337,191 describes in vitro protein synthesis using glycolytic intermediates as an energy source; and U.S. Pat. No. 6,168,931 describes enhanced in vitro synthesis of biological macromolecules using a novel ATP regeneration system.

### SUMMARY OF THE INVENTION

Compositions and methods are provided for the enhanced in vitro synthesis of nucleic acid molecules. A system is provided for the in situ phosphorylation of nucleoside monophosphates (NMPs) into nucleoside triphosphates (NTPs). This phosphorylation of NMPs in the reaction mix is driven by ATP. Also present in the reaction mix is an energy generating system for generation of ATP from AMP and ADP. Depending upon the ATP regeneration system chosen, use of this NTP supply system can prevent a net increase in free phosphate as a result of NTP hydrolysis, and also permits the use of relatively inexpensive nucleoside monophosphates in place of the triphosphates. Since the NMPs have a much lower affinity for Mg⁺⁺ than NTPs, the availability of free Mg⁺⁺ will remain more constant as the nucleotides are polymerized into nucleic acids. In order to permit the use of ATP to generate NTPs in situ, nucleotide kinase enzymes are also included in the reaction mix.

### BRIEF DESCRIPTION OF THE DRAWINGS

FIG. 1 provides a comparison of yields from in vitro transcription reactions using nucleoside triphosphates versus the nucleoside monophosphates.

FIG. 2 is a graph depicting the yield from a fed-batch in vitro transcription reaction.

### DETAILED DESCRIPTION OF THE EMBODIMENTS

Compositions and methods are provided for the enhanced in vitro synthesis of nucleic acid molecules. The methods of the invention utilize a reaction mixture comprising nucleoside monophosphates (NMPs), along with ATP and an energy system for recharging ATP from AMP and ADP. Triphosphosphates other than ATP are substantially absent from the starting reaction, although trace amounts will be present during the course of the reaction as the result of the ongoing phosphorylation reactions.

In vitro synthesis as used herein refers to the cell-free synthesis of nucleic acids in a reaction mix comprising biological extracts and/or defined reagents. The reaction mix will comprise at least ATP or ADP, an energy source and regenerative enzyme to generate ATP in situ; nucleoside monophosphates or deoxynucleoside monophosphates; a template for production of the macromolecule, e.g. DNA, mRNA, etc., and such co-factors, enzymes and other reagents that are necessary for the synthesis, e.g. transcriptional factors such as RNA polymerase, nucleoside monophosphate kinases, nucleoside diphosphate kinase, etc. Such enzymes may be present in the extracts used for transcription and translation, or may be added to the reaction mix. Such synthetic reaction systems are well known in the art, and have been described in the literature. The cell free synthesis reaction may be performed as batch, continuous flow, or semi-continuous flow, as known in the art. Synthetic systems of interest include the replication of DNA, which may include amplification of the DNA, and the transcription of RNA from DNA or RNA templates.

Phosphorylation of NMPs is driven by ATP, which is then converted to ADP. ADP is recharged to ATP by addition of an energy source. In order to permit the use of ATP to generate NTPs in situ, nucleotide kinase enzymes are included in the reaction mix. These methods result in significant cost savings, and provide a constant supply of nucleotide triphosphates in a manner similar to that in living cells. The initial chelation of Mg⁺⁺ is minimized, and phosphate accumulation is also minimized depending on energy system used, both of which factors allow the available magnesium ion concentration to be more precisely maintained. These features provide for increased rate and reaction duration. Typically Mg⁺⁺ is included in the reaction, at a concentration of at least about 2 mM, more usually at least about 6 mM, and preferably at least about 25 mM. The methods of the invention also provide for much higher Mg⁺⁺ concentrations, for example at greater than about 40 mM, greater than about 75 mM, and greater than about 100 mM. Generally the Mg⁺⁺ concentration will be less than about 250 mM, more usually less than about 200 mM.

The term nucleic acids, as used herein, is intended to refer to naturally occurring molecules, e.g. DNA or RNA, including DNA primers and longer sequences, tRNA, mRNA, rRNA, and synthetic analogs thereof, as known in the art. Analogs include those with modifications in the native structure, including alterations in the backbone, sugars or heterocyclic or non-native bases. The nucleic acids thus generated find use in a variety of applications. For example, RNA is useful as ribozymes, translational templates, tRNA molecules, RNAi and antisense therapeutics. DNA is useful for vaccines, for gene therapy, as an expression template for cell-free protein synthesis, and the like.

The methods of the invention find particular use in coupled reactions of transcription and translation, for protein synthesis with eukaryotic cell extracts. Most eukaryotic in vitro protein synthesis systems require low magnesium concentrations for efficient translation. However, these low magnesium concentrations then result in inefficient messenger RNA synthesis from NTPs. The present invention allows the production of mRNA from NMPs, which have a lower affinity for magnesium. This system allows effective mRNA synthesis at the low magnesium concentrations required for eukaryotic translation.

The methods of the invention mimic in some ways the in vivo environment for transcription, in which a nucleotide species supplies the nucleotides for incorporation into RNA through phosphorylation of nucleotide monophosphates to nucleotide diphosphates and then another to nucleotide triphosphates. Both sets of reactions are catalyzed by the appropriate kinase enzymes, utilizing energy supplied by ATP.

During in vitro transcription, various sources may be used to generate ATP, for example by using high-energy phosphate carbon molecules that donate a phosphate bond to ATP. These include phosphoenol pyruvate (PEP), creatine phosphate, and acetyl phosphate, in combination with the enzymes pyruvate kinase, creatine kinase and alkaline phosphatase, respectively. The appropriate enzyme is included in the reaction mixture in an effective amount.

In the phosphorylation of an NMP to the corresponding NDP, one ATP equivalent is consumed. A specific kinase for each of the four nucleotides catalyzes the reaction, e.g. adenylate kinase, CMP kinase, guanylate kinase, UMP kinase. UMP kinase has sufficient affinity for cytidine monophosphate that CMP kinase is not necessary, and guanylate kinase has sufficient affinity for adenosine monophosphates that adenylate kinase is not necessary. These enzymes are included in the reaction mixture in an amount effective to catalyze the reactions.

A single nucleotide diphosphate kinase converts all of the nucleotide diphosphates to their nucleotide triphosphates, which each reaction consumes one ATP equivalent. This enzyme is included in the reaction mixture in an effective amount.

When the nucleic acid polymer is DNA, the reaction mixture is modified to comprise deoxyribonucleotide monophosphates and enzymes required for phosphorylation. Alternatively, ribonucleotide monophosphates and the phosphorylation enzymes are employed, along with a ribonucleotide reductase to convert ribonucleoside diphosphates to deoxyribonucleoside diphosphates. The ribonucleotide reductase system requires a supply of NADPH, using a chemical source or reduction potential and may also require such factors as thioredoxin and glutaredoxin and their reductase catalysts. Where the DNA synthesis utilizes polymerase chain reaction, it is desirable to use thermostable enzymes. Where the desired nucleic acid is an analog of DNA or RNA, the appropriately modified nucleoside(s) are included in the reaction mixture.

ATP may be regenerated by a variety of mechanisms, for example see U.S. Pat. Nos. 6,337,191 and 6,168,931, herein incorporated by reference. In one embodiment of the invention, a high phosphate bond molecule is used as an energy source, for example PEP, creatine phosphate, acetyl phosphate, and the like, for example phosphoenolpyruvate (PEP) along with the enzyme pyruvate kinase. Generally, a small amount of ATP or ADP is present for initializing the reactions. In a similar manner, pyrophosphatase may be included in the reaction mixture, particularly when the phosphate is being recycled, e.g. when pyruvate is the energy source. Polyphosphate also finds use in recycling ATP. The concentration of energy sources is usually at least about 1 mM, more usually at least about 2 mM, and may be 10 mM or higher. Usually the energy source will be present at less than about 100 mM, more usually less than about 50 mM.

In another embodiment, pyruvate is used as the energy source in combination with the enzyme pyruvate oxidase, EC 1.2.3.3.; CAS: 9001-96-1. It is known that pyruvate oxidase is produced by a variety of microorganisms. For example, it is known to be produced by Lactobacillus delbrueckii, Lactobacillus plantarum, microorganisms of the genus Pediococcus, Streptococcus, and Aerococcus, microorganisms of the genus Leuconostoc, etc. During oxidation of pyruvate, acetyl phosphate is generated, which then directly regenerates ATP from ADP via the catalytic activity of the enzyme acetate kinase. The by-product hydrogen peroxide is converted to water and oxygen by the action of the enzyme catalase or another peroxidase. The phosphate that is hydrolyzed from ATP is recycled during the pyruvate oxidation to generate acetyl phosphate, thereby preventing a net accumulation of free phosphate, which can have an inhibitory effect on synthetic reactions. Pyruvate may be supplied as a suitable biologically acceptable salt, or as the free acid, pyruvic acid. The final concentration of pyruvate at initiation of synthesis will usually be at least about 1 mM, more usually at least about 10 mM, and not more than about 500 mM, usually not more than about 100 mM. Additional pyruvate may be added to the reaction mix during the course of synthesis to provide for longer reaction times.

Any of the required enzymes can be provided for in the reaction mix in a variety of ways. Purified or semi-purified enzyme may be added to the reaction mix. Commercial preparations of the enzymes described above are available, or the enzyme may be purified from natural or recombinant sources according to conventional methods. For example, the genetic sequences of pyruvate oxidases, pyruvate kinase, creatine kinase, etc. may be used as a source of recombinant forms of the enzyme. In the case of coupled transcription and translation reactions, the enzymes may also be included in the extracts used for synthesis. For example, extracts can be derived from E. coli for protein synthesis. The E. coli used for production of the extracts may be genetically modified to encode suitable enzymes. Alternatively, where the synthetic reactions include protein synthesis, a template, e.g. mRNA encoding the desired enzyme, or a plasmid comprising a suitable expression construct, etc. may be spiked into the reaction mix, such that a suitable amount of enzyme is produced during synthesis.

The reactions may utilize a large-scale reactor, small scale, or may be multiplexed to perform a plurality of simultaneous syntheses. Continuous reactions will use a feed mechanism to introduce a flow of reagents, and may isolate the end product as part of the process. Batch systems are also of interest, where additional reagents may be introduced to prolong the period of time for active synthesis. A reactor may be run in any mode such as batch, extended batch, semi-batch, semi-continuous, fed-batch and continuous, and which will be selected in accordance with the application purpose.

In addition to the above components such as cell-free extract, genetic template, nucleotide monophosphates and energy sources, materials specifically required for synthesis may be added to the reaction. These materials include salt, polymeric compounds, cyclic AMP, inhibitors for nucleic acid degrading enzymes, oxidation/reduction adjuster, non-denaturing surfactant, buffer component, spermine, spermidine, etc.

The salts preferably include potassium, magnesium, ammonium and manganese salt of acetic acid or sulfuric acid, and some of these may have amino acids as a counter anion. The polymeric compounds may be polyethylene glycol, dextran, diethyl aminoethyl, quaternary aminoethyl and aminoethyl. The oxidation/reduction adjuster may be dithiothreitol, ascorbic acid, glutathione and/or their oxides. Also, a non-denaturing surfactant such as Triton X-100 may be used at a concentration of 0-0.5 M.

When changing the concentration of a particular component of the reaction medium, that of another component may be changed accordingly. For example, the concentrations of several components such as nucleotides and energy source compounds may be simultaneously controlled in accordance with the change in those of other components. Also, the concentration levels of components in the reactor may be varied over time.

Preferably, the reaction is maintained in the range of pH 5-10 and a temperature of 20°-50° C., and more preferably, in the range of pH 6-9 and a temperature of 25°-40° C.

In vitro transcription reactions have a number of uses. One use is the synthesis of high specific radioactivity RNA probes, using radioactively labeled nucleotides as substrates. Another is the synthesis of larger amounts of unlabeled RNA for a variety of molecular biological uses that may benefit greatly by the use of the reaction mixture disclosed herein. These include, but are not limited to, in vitro translation studies, antisense RNA experiments, microinjection studies, and the use of RNA in driving hybridization reactions for the construction of subtractive cDNA libraries and the like. In particular, when very large libraries are constructed using in vitro techniques, the cost and efficiency of these reactions is critical.

It is to be understood that this invention is not limited to the particular methodology, protocols, cell lines, animal species or genera, constructs, and reagents described, as such may, of course, vary. It is also to be understood that the terminology used herein is for the purpose of describing particular embodiments only, and is not intended to limit the scope of the present invention, which will be limited only by the appended claims.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing, for example, the cell lines, constructs, and methodologies that are described in the publications, which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

The following example is put forth so as to provide those of ordinary skill in the art with a complete disclosure and description of how to make and use the subject invention, and is not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

As used herein the singular forms “a”, “and”, and “the” include plural referents unless the context clearly dictates otherwise. Thus, for example, reference to “a cell” includes a plurality of such cells and reference to “the protein” includes reference to one or more proteins and equivalents thereof known to those skilled in the art, and so forth. All technical and scientific terms used herein have the same meaning as commonly understood to one of ordinary skill in the art to which this invention belongs unless clearly indicated otherwise.

### EXPERIMENTAL

### Example 1

### In-Vitro Transcription and Other Polynucleotide Synthetic Reactions Using Nucleoside Monophosphates

Methods and Materials

Chemicals were purchased from Sigma (St. Louis, Mo.) except phosphoenolpyruvate from Roche, and dithiothreitol from Gibco. Enzymes were purchased from Sigma except NMP kinase from Roche. RNaseOUT from Invitrogen, and T7 RNA polymerase was made and purified in our lab according to protocols described elsewhere. (Davenloo et al.)

DNA templates were prepared from PCR of genomic E. coli DNA strain A19. Utilizing primer extension to add on the −17 consensus sequence of the T7 RNA promoter region to make the rare transfer RNAs found in E. coli: Arg U, Arg W and Leu W.

DNA produced from PCR based reactions were directly used for in vitro transcription reactions except in the case of optimization experiments. For these experiments the DNA was purified using phenol/chloroform extraction followed by precipitation with 0.8 M LiCl and ethanol. The purified DNA was resuspended in TE Buffer (10 mM Tris, 1 mM EDTA).

In-vitro transcription reaction conditions. For a standard transcription reaction with NTPs the following concentrations were used: 80 mM Hepes, 25 mM magnesium acetate, 20 mM DTT, 2 mM spermidine, 20 nM DNA, 6 mM of ATP, CTP, GTP, and UTP; 2.5 μl/100 μl RnaseOUT in 20-200 μl reactions using 0.1% DEPC water to mix reagents.

For transcription using NMPs the following concentrations were used: 80 mM Hepes, 25 mM magnesium acetate, 20 mM DTT, 2 mM spermidine, 20 nM DNA, 6 mM of AMP, CMP, GMP, and UMP; 2 mM ATP, 5 mM PEP, 0.5 mg/ml NMP kinase, 1 U guanylate Kinase, 123 U pyruvate kinase, 0.5 U NDP kinase, 2.5 μl/100 μl RnaseOUT in 20-200 μl reactions using 0.1% DEPC water to mix reagents. The mixture for both NTPs and NMP conditions are incubated in a water bath at 37° C. for 3 hours.

Samples were heated for 10 minutes at 85° C. prior to analysis in order to breakup secondary structure of the RNA.

HPLC Quantification. Quantification of RNA produced was done using an Agilent ChemStation 1100 HPLC equipped with a diode array detector. A Dionex DnaPac column was utilized using running buffers of 5 M Urea, 25 mM Tris-Cl pH 7.8 and a gradient from 0 to 2 M NaCl. A flow rate of 1 ml/min was used. The peak area was taken from a chromatogram at 260 nm.

Results

The in vitro transcription using NMPs instead of NTPs had a slight yield improvement after both reaction conditions were optimized for the length of DNA template being used. See Table 1 for optimization parameters. A standard fractional factorial design with centerpoints was performed using DNA templates as a blocked variable. After a first round of optimization the only significant parameters are the concentration of magnesium and the concentration of nucleotide. These were also highly correlated to each other. A second round of optimization used the concentrations of reagents that tended to yield the least amount of non-full length transcript. The second round of optimization used the prior value as center and took range around that including star points with an alpha value of 1.4.

TABLE 1

| Reagent | Concentration range | |
|---|---|---|
| Hepes | 40-400 | mM |
| Mg ions | 5-40 | mM |
| DTT | .04-20 | mM |
| Spermidine | 0-20 | mM |
| NTP | 1-11 | mM |
| NMP | 1-11 | mM |
| PEP | 2-11 | mM |
| NMP kinase | .1-1 | mg/ml |
| Guanylate kinase | .15-1.5 | U |
| NDP kinase | .25-2.5 | U |
| DNA | 1-20 | ng |
| T7 RNA polymerase | | |

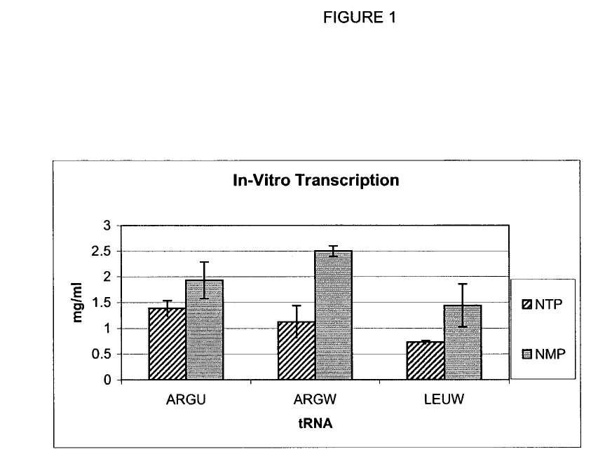

A comparison of optimized reactions for utilization of NTPs versus NMPs is shown in FIG. 1. This shows that for all three templates used, there is a significant yield increase when using NMPs instead of NTPs. It is also evident that there is variation in transcriptional efficiencies based on the DNA template used.

The system provides for a significant reduction in the expense of some reagents. Outside of the cost of RNAse inhibitor (a reagent to prevent degradation from Rnases that are difficult to keep out of the laboratory), nucleotides make up the major cost in a conventional reaction. The NMPs are significantly less expensive than the NTPs. Although there are additional costs for the kinase enzymes, these can be produced by recombinant methods. A major commercial advantage of the system of the invention is a reduction in substrate cost, and improvement in yield. The yield improvement has further impact beyond savings in reagent costs, because higher yields per batch contribute to reduced labor and equipment costs in production to produce the same amount of product.

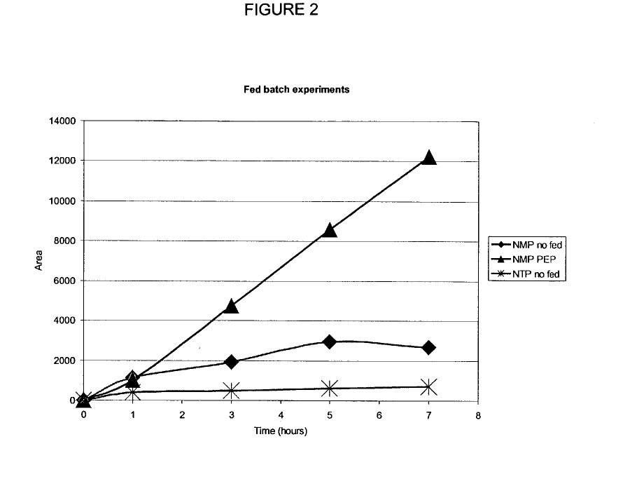

A fed batch experiment was performed. This consisted of feeding the reactions with PEP every other hour: 2, 4, 6, and 8. The amount of PEP added was equal to the initial amount added at time zero. As shown in FIG. 2, the rate of the reaction is maintained in the fed batch reaction and produces a larger amount of RNA compared to the batch reaction. Another fed batch experiment used PEP to feed the NMP reaction and NTPs to feed the NTP reaction (1.67 mmol/ul reaction using 125 mM NTP mix). In addition magnesium acetate was added to maintain the ratio of nucleotides to magnesium ions.

The use of NMPs shows an advantage over the use of NTPs, in both a yield improvement and a potential cost improvement. The homeostatic conditions achieved using NMPs is illustrated by being able to maintain the initial reaction rate for several hours.

What is claimed is:

1. A method for in vitro synthesis of nucleic acids in a reaction mix, the method comprising:
generating nucleoside triphosphates from nucleoside monophosphates by catalysis with nucleoside monophosphate kinase, nucleoside diphosphate kinase and ATP;
generating ATP from an energy source using an ATP generating enzyme; and
synthesizing said nucleic acids in said reaction mix utilizing said nucleoside triphosphates.

2. The method of claim 1, wherein nucleic acid is RNA.

3. The method of claim 1, wherein said nucleic acid is DNA and said nucleoside monophosphates are deoxyribonucleoside monophosphates.

4. The method according to claim 1, wherein said nucleic acid is DNA, said nucleoside monophosphates are ribonucleoside monophosphates, and wherein said reaction mix further comprises ribonucleotide reductase and a source of NADPH and other required cofactors.

5. The method according to claim 1, wherein said energy source is pyruvate and said regenerative enzyme is pyruvate oxidase in conjunction with acetate kinase and an enzyme with catalase activity.

6. The method according to claim 1, wherein said energy source is phosphoenol pyruvate and said regenerative enzyme is pyruvate kinase.

7. The method of claim 2 wherein said synthesis comprises transcription of mRNA from a DNA template.

8. The method of claim 1 wherein said synthesis is performed as a batch reaction with or without subsequent additions.

9. The method of claim 1, wherein said synthesis is performed as a continuous reaction.

10. The method according to claim 1, wherein said energy source is selected from the group consisting of phosphoenol pyruvate, pyruvate, acetyl phosphate, creatine phosphate, and pyrophosphate.

11. The method according to claim 1, wherein said nucleic acid is a mixture of DNA and RNA and said nucleoside monophosphates comprise deoxyribonucleoside monophosphates, ribonucleoside phosphates, and derivatives thereof.
